(19) United States
(12) Patent Application Publication
(10) Pub. No.: US 2010/0312312 A1
(43) Pub. Date: Dec. 9, 2010

(54) METHOD FOR SELECTIVE PHOTODYNAMIC THERAPY AND LIGHT SOURCE FOR IMPLEMENTATION THEREOF

(76) Inventor: Gary Wayne Jones, Newcastle, WA (US)

Correspondence Address: SCHMEISER, OLSEN & WATTS, 22 CENTURY HILL DRIVE, SUITE 302, LATHAM, NY, 12110, US

(21) Appl. No.: 12/797,170

(22) Filed: Jun. 9, 2010

Related U.S. Application Data
(60) Provisional application No. 61/185,346, filed on Jun. 9, 2009.

(51) Int. Cl. A61N 5/06; A61K 39/395; A61K 35/76; A61P 35/00; A61P 31/00; A61P 3/04; A61P 9/10

(52) U.S. Cl. 607/88; 424/178.1; 424/93.6

(57) ABSTRACT

Disclosed is a method of photodynamic therapy that includes introducing a selective photocytotoxic compound to a body having a target cell, wherein the selective photocytotoxic compound is configured to selectively attach to or enter the target cell. The method further includes activating the selective photocytotoxic compound with a light source. Further disclosed is a method that includes introducing a selective photoluminescent compound to a body having a target material. The selective photoluminescent compound is configured to selectively attach to or enter the target material. The method includes introducing an activating light to the selective photoluminescent compound, wherein the photoluminescent compound is configured to absorb the activating light and emit an emission light having a different wavelength than the activating light for diagnosis and locating diseased areas. The method further includes activating a photocytotoxic compound with the emission light of the selective photoluminescent compound. A novel light source is further disclosed.

### RELATED APPLICATION

This non-provisional application claims priority to a U.S. Provisional Application Ser. No. 61/185,346 filed Jun. 9, 2009 with the United States Patent and Trademark Office.

### FIELD OF TECHNOLOGY

The subject matter disclosed herein relates generally to cancer or pathogen treatment, and other type cells or materials selectable using this new methodology. More particularly, the subject matter relates to a photodynamic therapy for the selective and localized reactivity with cells, bacteria, fungi, and other materials.

### BACKGROUND OF TECHNOLOGY

Current chemotherapy, gene therapy and pathogen treatments typically require the entire body be exposed to the active cancer or pathogen killing materials, providing opportunities for other cells of various types in the body to be adversely effected. Therefore, concentrations of aggressive compounds for fighting cancer or pathogens are usually kept low to minimize serious side effects. Low dosages often give the cancer or pathogen a better chance to survive and multiply. Additionally, low dosage treatments are frequently long-term and are associated with undesirable, sometimes lethal, side effects. Selective methods for such prior chemically reactive drugs have all exhibited some non-selectivity, affecting other parts of the body, especially the liver and kidneys where such compounds tend to concentrate. Even small amounts of non-selectivity or partially misdirected selectivity may be dangerous to a patient.

Prior photodynamic cancer or pathogen treatment work almost like a scalpel, but minimizes the incision size. In this existing treatment, a light source activates photosensitive reactive compounds that kill all cells where specific wavelength(s) are absorbed which originate from the light source. This method results in a destruction of almost all the cells that the light source exposes. Some concentration of the photo-reactive chemicals may occur at tumor site, but it may not provide >200 times as selective to cancer cells or pathogens as desired. Although cancer or pathogen cells are typically physically exposed with the light source or the source is centered in the tumor or immediately on the tumor, it often occurs that a great amount of normal tissue in the vicinity of the cancer or pathogen cells are destroyed in order to reasonably insure the targeted cancer or pathogen cells are also eliminated. Furthermore, some of the cancer cells or pathogens that have migrated from the target site may be completely missed, enabling reemergence of the disease.

Thus, a photodynamic therapy treatment that more accurately localizes the destruction of target cells while potentially covering a broader area of treatment, and thereby reducing the destruction of normal cells may be well received in the art.

### SUMMARY

According to one embodiment, a method of selective photodynamic therapy comprises: introducing a selective photocytotoxic compound to a body having a target cell, wherein the selective photocytotoxic compound is configured to at least one of selectively attach to and selectively enter the target cell; and activating the selective photocytotoxic compound, at least one of directly and indirectly, with a light source.

According to another embodiment, a method of selective photodynamic therapy comprises: introducing a selective photoluminescent compound to a body having a target cell, wherein the selective photoluminescent compound is configured to at least one of selectively attach to and selectively enter the target cell; introducing an activating light to the selective photoluminescent compound, wherein the photoluminescent compound is configured to absorb the activating light and emit an emission light having a different wavelength than the activating light; and activating a photocytotoxic compound with the emission light of the selective photoluminescent compound.

According to yet another embodiment, a light source comprises: a light pathway configured to transmit a light of a first wavelength; and a tip section having a photoluminescent material located along the light pathway, the light of the first wavelength configured to be received by the photoluminescent material of the tip section and emitted from the light source as an emitted light having a second wavelength.

### BRIEF DESCRIPTION OF THE DRAWINGS

The subject matter which is regarded as the invention is particularly pointed out and distinctly claimed in the claims at the conclusion of the specification. The foregoing and other features and advantages of the invention are apparent from the following detailed description taken in conjunction with the accompanying drawings in which:

FIG. 1(a) depicts a submicroscopic depiction of a selective photoluminescent compound having a plurality of photoluminescent compounds and a bacteriophage bonded together with a chemical bond;

FIG. 1(b) depicts a submicroscopic depiction of a selective photodynamic compound having both a plurality of photoluminescent compounds and photocytotoxic compounds and a bacteriophage bonded together with a chemical bond;

FIG. 1(c) depicts a submicroscopic depiction of a selective photodynamic compound having a plurality of photoluminescent compounds and photocytotoxic compounds bonded together and also bonded with a bacteriophage;

FIG. 1(d) depicts a submicroscopic depiction of a selective photocytotoxic compound having a plurality of photocytotoxic compounds and a bacteriophage bonded together with a chemical bond;

FIG. 2 depicts a filtering process of bacteriophage tags with target cells in a medium according to one embodiment;

FIG. 3 depicts a filtering process of bacteriophage tags with target cells in a medium according to one embodiment;

FIG. 4 depicts a representation of a target cell with selective pages attached to the cell and phages in the vicinity that are likely to attach to the target cell according to one embodiment;

FIG. 5 depicts a method of treatment utilizing a selective compound according to one embodiment;

FIG. 6 depicts a chemical composition of talaporfin sodium;

FIG. 7 depicts a “step” method of treatment utilizing a plurality of PL compounds according to one embodiment;

FIG. 8 depicts a method for imaging using the selective PL compounds according to one embodiment;

FIG. 9 depicts a light source probe for photodynamic therapy according to one embodiment;

FIG. 10 depicts another light source probe for photodynamic therapy according to one embodiment;

FIG. 11 depicts a light source probe having a flexible tip according to one embodiment; and

FIG. 12 depicts a scanning apparatus according to one embodiment.

### DETAILED DESCRIPTION

A detailed description of the hereinafter described embodiments of the disclosed apparatus and method are presented herein by way of exemplification and not limitation with reference to the Figures.

Disclosed herein is a selective photodynamic compound that locates a target cell in a body and attaches to that target cell and thereafter performs a function when exposed to a light source of a particular wavelength. The selective photodynamic compound generally comprises a photodynamic compound bonded with a bacteriophage virus, monoclonal antibody, other transporter material, or other tag. Landscape filamentary bacteriophage virus strands appear to be especially useful for this application, although other phage types may also be used. It should be understood that a photodynamic compound may be either a photoreactive compound such as a photocytotoxin (PCT) compound, or a photoluminescent (PL) compound. A photoreactive compound reacts chemically to the exposure of light. A PCT compound, for example, releases a reactive oxygen singlet when exposed to a light spectrum capable of activating the compound. A PL compound absorbs photons and then re-radiates photons of a different wavelength. Some compounds such as squaraines may be made to act only as a non-reactive PL material or also as a PCT. Most PCT compounds also have PL characteristics. Examples of photodynamic compounds include porfiner compounds, talaporfin compounds, squaraines, gadolinium complex compounds, and the phthalocyanide (e.g., zinc phthalocyanide) compounds. Examples of PL compounds are squaraines, polymethines, and xanthene dyes. Water solubility is a strong asset for biological applications, particularly when the PL or PCT molecules are attached to a phage viral strand. Minimally-agglomerating nanoparticles and/or nanocrystals may also be used. These nanoparticles and/or nanocrystals may be made of non-water soluble dyes such as perylene-tetracarboxylic dyes. Viral strands of landscape bacterial phage may have many PL and/or PCT molecules attached to each strand. The practical limit may be over 10% of the potential bonding sites or well over 400 molecules per viral strand without significant selectivity loss. This may also vary with the type phage, characteristics of the selective target cell or pathogen, desired selectivity, probability of linking to a target cell or pathogen, immune response probabilities, and other factors. The practical limit may be experimentally determined for most cases by determining the number of molecules per phage strand that the selectivity or probability of establishing a link to the target cells of pathogens falls off enough to reduce the effectiveness of the therapy or analysis. Some photocytotoxic compounds 13, such as talaporfin, may even be linked together and on other carriers to permit additional concentrations of active chemicals on each viral strand.

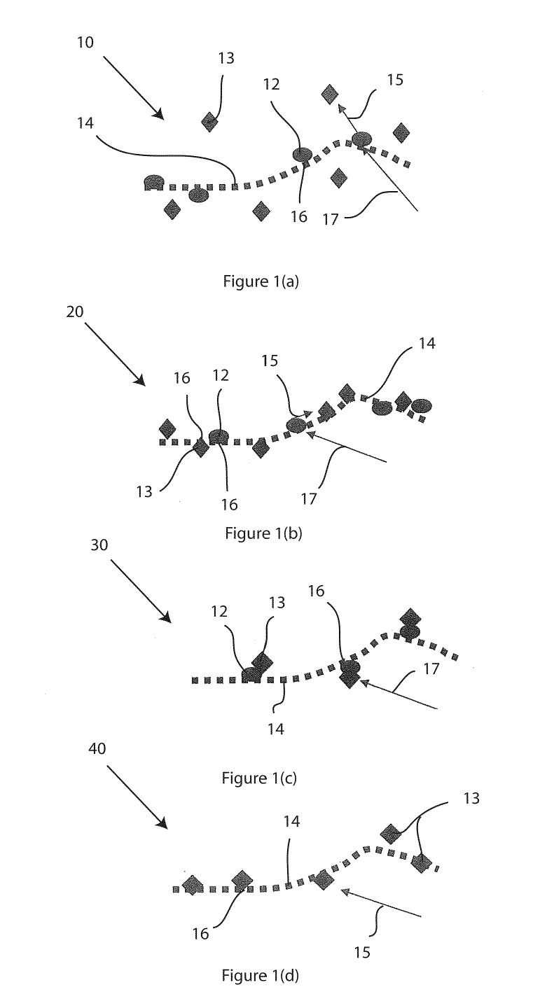

Referring first to FIG. 1(a), a microscopic depiction of a selective PL compound 10 is shown having a plurality of PL compounds 12 and a bacteriophage (hereinafter referred to interchangeably as a phage) 14 bonded together with a chemical bond 16. It should be understood that the selective PL compound 10 may instead be a single PL compound 12 attached to a single bacteriophage 14. A PCT compound 13 is shown in the vicinity of the selective PL compound 10. The PCT compound 13 may, for example, be located in a fluid near targeted cells (not shown). In any case, the PCT compound 13 may be activated by a PCT activating spectrum of light 15. In this embodiment, the spectrum of light 15 is emitted by the PL compound 12. On the other hand, the PL compound 12 may be activated by a PL activating spectrum of light 17. The spectrum of light 17 may cause the PL compound to emit the spectrum of light 15. The bacteriophage 14 is configured to preferentially attach to a targeted cell or enter targeted cells, as will be described hereinafter, while the PCT compound 13 is configured to release at least one singlet oxygen (not shown) or other short duration cytototoxic element or compound when exposed to the particular spectrum of light 15. The selective PL compound 10 thereby both selectively links to a targeted cell (such as the cell 24 shown described herein below), and emits light 15 when exposed to the spectrum of light 17. The emitted light 15 may then locally activate nearby PCT compounds 13 and create at least one singlet oxygen from residual dissolved oxygen in the body, or other cytotoxic element or compound when exposed to the activating light 15. The cytotoxic chemical, or PCT compound 13, induces cell destruction to nearby cells or may react with biological materials. The characteristic light spectrum 15 emitted by the PL compounds 12 may also be used to identify the selective phage type 14 and, identify where is has concentrated, or identify if the phages employed have found the target cells, pathogens, or other biological materials. It should be understood that carriers such as monoclonal antibodies or bacteriophage 14 is used by way of exemplification but that this is not limiting, as other photoluminescent (PL) tags may be used in a similar manner. It should also be understood that a single bacteriophage 14 may have a plurality of the PL compounds 12 attached thereto. By way of exemplification, it is contemplated that tens, hundreds, or thousands of PL compounds 12 may be attached to each carrier such as a bacteriophage 14. Further, a plurality of tens, hundreds, or even thousands of carriers such as monoclonal antibodies or bacteriophages 14 may be configured to attach to a single of the targeted cell, as will be described hereinbelow.

Referring now to FIG. 1(b), a microscopic depiction of a selective photodynamic compound 20 is shown having both a plurality of PCT compounds 13 and a plurality of PL compounds 12 attached to carriers such as a bacteriophage 14. The PCT compounds 13 and the PL compounds 12 may be bonded together with the bacteriophage 14 with a chemical bond 16. The carriers such as bacteriophage 14 is configured to preferentially attach to a targeted cell, as will be described hereinafter, while the PCT compound 12 is configured to create or release at least one singlet oxygen, or other short duration cytotoxic element or compound, when exposed to a particular spectrum of light. The selective photodynamic compound 20 thereby selectively links to a targeted cell. A first light emission, such as the emission 17, may then be introduced to the selective photodynamic compound 20, activating the PL compound 12 to emit a second light emission, such as the emission 15. The second light emission 15 may then trigger the PCT compound 13 to create or release at least one singlet oxygen or other cytotoxic element or compound. Such a compound, such as a reactive version of chlorin e6, talaporfin, or Indocyanine green (ICG) may continually generate a cytotoxic chemical such as singlet oxygen as long as light energy of the appropriate spectrum is provided or until the material itself degrades, or in some cases the photocytotoxic compound itself may directly release the cytotoxin. The cytotoxic chemicals 13 induce cell destruction to nearby cells or may react with to other biological materials. In a similar manner as described hereinabove with respect to FIG. 1(a), a plurality of the PCT compounds 13 and PL compounds 12 may be attached to each phage 14. Further, a plurality of phages may be configured to attach to each target cell. For example, chlorin e6 or talaporfin generate singlet oxygen upon exposure to red spectrum light with a peak absorption at 664 nm.

In FIG. 1 (c), the PCT compounds 13 are bonded together with the PL compounds 12 to form another selective photodynamic compound 30. The combination of the PCT compound 13 and the PL compound 12 are each shown bound to a selective phage 14. It is possible to chemically link the PCT compound 13 or the PL compound to the selective targeting phage 14 in this way to allow the phages 14 to bring both the PL and PCT materials 12, 13 to the targeted areas. This may be used to shift the spectrum sensitivity of the PCT compound 13 and/or provide additional spectrum emission. Förster resonance energy transfer (abbreviated FRET), may also be used to absorb shorter wavelength energy and transfer the energy to a longer wavelength chromaphore in a molecular conjugate. Since some of the PCT and PL compounds 12, 13 have multiple bonding sites, it is possible to have multiple PCT and/or PL molecules 12, 13 of the same or different types bound together. It is also possible to have mixed PCT and PL compounds 12, 13 bound together. It is also possible to bond PCT and PL compounds together using additional molecules as conjugates to increase the concentrations or provide additional light activation mechanisms. The attached PL molecules may be used to activate the PCT compound 13. Alternately, the PL molecules may be simply used as tags that are independent of the PCT compound 13. Different carriers such as monoclonal antibodies or bacteriophage with different PL and PCT compounds 12, 13 may be prepared and mixed that may concentrate at the targeted cells, pathogens, or other targeted material(s) 24. This technique may also be used to shift the emission spectrum to increase the amount and type of toxins released from the PCT compound 13 upon exposure to the activating light, to carry other molecules of use to the target site, or any combination of these and other potential benefits. This can be useful with broad spectrum light sources as long as the shorter wavelengths used still sufficiently penetrate the tissue depth desired to activate the target materials.

In FIG. 1(d), a selective PCT compound 40 is shown. The selective PCT compound 40 includes only the PCT compound 13 being attached to the selective phage 14. The selective PCT compound 40 then concentrates at the targeted areas when introduced to a body and is then activated directly with an incoming spectrum of light 15. Many PCT materials 13 not only release a cytotoxic chemical upon activation. The PCT itself may also be photoluminescent with an identifiable characteristic emission spectrum. This may help provide feedback on the location of the selective PCT compound 40 or the phage 14 and PCT compound 12 locations of concentration.

It should be understood that the PL material 12 may also be used only for spectral emission tagging and identification using the same or different spectra incoming light as the incoming light that is used to separately activate the PCT compound 13, instead of and/or in addition to using the PL emission spectrum to activate the PCT. The photoluminescent emission from the PL molecules 12 and many PCT compounds 13 may provide a mechanism for identifying if the phages 14 have found the target cell, pathogen, or target material. The photoluminescent emission 15 from the PL molecules and many PCT compounds may provide a mechanism for identifying if the phage have found the target cell, pathogen, or target material and concentrated, as well as where concentration(s) have occurred. Furthermore, different PL compounds 12 or PCT compounds 13 attached to selective phage 14 may provide an identification of the type of pathogen present if different identification compounds are linked to selective phage with different targets (diagnosis and analysis).

In all embodiments of FIGS. 1(a) -1(d), the PCT or other reactive chemical processes are similarly ideally localized to the area near the targeted cells, pathogens, or other biological material and to where a suitable activating light spectrum is provided. Further, the characteristic emission of the PL and/or the PCT materials 12, 13 may be used for identification and for locating areas of selective phage 14 concentration using optical sensor(s) such as spectrometers. Still further, it should be recognized that the bacteriophage 14 is used by way of exemplification. However, this is not limiting, as other tags, PCT materials, and types of phage may be used in a similar manner. Furthermore, in all the embodiments, many PCT and/or PL compounds 12, 13 may be attached to a single phage 14, and many phage 14 may attach to a target cell, pathogen, or other target material 24 permitting high concentrations of PL tags and/or PCT compounds 12, 13 to be directed to target sites.

In order to make sure that the selective PCT compounds 10, 20, 30, 40 target the desired type of cell, an appropriate washing and filtering process must be used. Using a filtering process, it is possible to remove any phages that did not attach to the targeted cells, pathogens, or biomaterials. The one or more of the many millions or billions of strands of bacteriophages that are attracted to a particular target cell, pathogen, or biological material such as a cancer cell, bacteria, arterial plaque, or the like, are retained. The particular strand or strands of carrier such as phage that are attracted to the target cells or biomaterials are then allowed to multiply (amplify) on or in a bacterial medium such as a nonpathogenic e-coli. After this amplification process, a large number of this particular phage is created. Then, an optional but useful repeat of the above process may be performed to increase the probability of attachment during later diagnosis or treatment. After the filtration process, it is then contemplated to bond that particular type of phage 14 to the PCT and/or PL compound(s) 12, 13.

Optionally in the case of bacteriophage (phage), the above process, using the amplified phages attracted to the targeted cells, may be used with a high concentration of normal cells similar to those near the planned treatment site. This may help prevent phages from being used that are also attracted to the normal cells. Again, this process may be repeated in any reasonable order as needed to obtain highly selective phages. The more different the target material is from the normal cells, the fewer repeat cycles may be necessary to obtain high selectivity. Again, after the filtration process, it is then contemplated to bond that particular type of phage 14 to the PCT and/or PL compound(s) 12, 13.

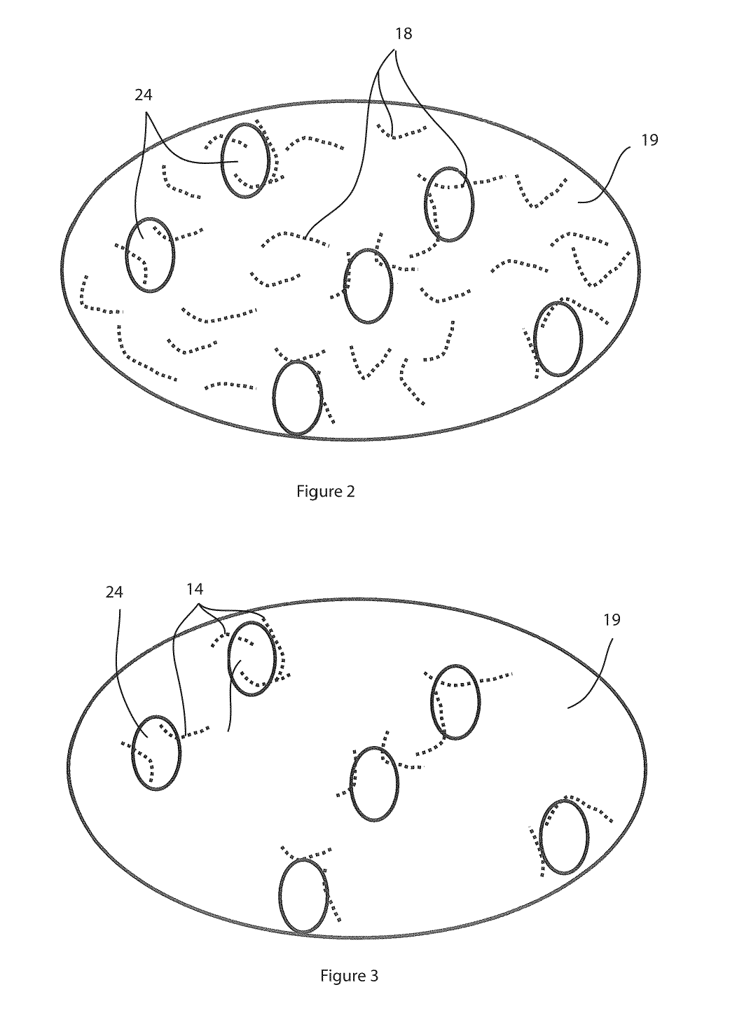

An example of the selection and filtering process is shown in FIGS. 2-3. First, the process includes taking a sample biopsy of target cells 24 or target material from a patient. Referring to FIG. 2, the target cells 24 are spread on a growth medium or container 19. Next, a large library of many millions or billions of bacteriophage virus strands 18 from a prepared grouping (these groupings are also called library) of phage known to have a reasonable probability of some of the phage library attaching to the targeted type material may be spread/mixed onto/into the biopsy or sampling of the target cells, pathogens, or other targeted bio-material 24. The bacteriophage strands that do not attach to the particular target cells 24 may then be washed and removed, while one or more remaining strands 14 are left attached to some or all of the target cells 24, as shown in FIG. 3. These phages 14 may thereby have a tendency to preferentially attach to the desired target material 24, cells, or pathogens and are therefore referred to as a “selective phage” herein. Next, the target cells, pathogens, or other targeted biomaterial 24 are reactively removed leaving only the particular selective phage strands 14 that were attached to the target cells 18. The particular selective phage 14 may then be multiplied using common and safe bacteria as their food. The selective phage 14 may then be purified. Next, the selective phage 14 may be applied into another high concentration spread of the normal cells 25 to make sure that the particular strand of the bacteriophage 14 will not attach to the normal cells as well. In this process, the target selective phages 14 that may also be attracted to normal cells 25 of various types are removed prior to subsequent amplifications. In this process, the washed selective phages 14 that did not attach to the normal cells 25 are retained. Normal cells 25 may be obtained from a single subject or multiple subjects and from multiple types of tissue, cells, or other non-targeted materials as may be relevant to minimize attraction to the non targeted cells and materials that may be in the vicinity of targeted materials during subsequent light activation of the PCT compounds 13 or photoluminescent analysis or diagnosis. The attraction to target cells or target material 24 and the non-attraction to normal cell and non-targeted materials 25 selection and filtering processes may be repeated in any reasonable order or sequence as necessary to increase the selectivity of the phage 14 to the desired target cells, pathogens, or other target biomaterials 24. For example, one or more of the selective phages 14 may attach the target cells 24, but sometimes also to the normal cells 25. The chances of this happening may be about 1 time in every 300 times (this may, of course, be higher or lower). As the number of selective phage separation and purification processes increases, fewer normal cells may be tagged by the phages. Also as phage technology and libraries of phage may improve, fewer normal cells may be tagged by the phages. Thus, more phages each carrying higher concentrations of PL and PCT molecules may thereby tag target cells and pathogens.

Once it is confirmed that the bacteriophage 14 will attach with adequate selectivity to the target cells 24, the bacteriophage 14 may then be bonded with the PCT or PL compounds 12, 13 with the bond 16. The bond 16 may occur because both bacteriophages and the selected photodynamic compounds have various carboxyl groups or other chemical bonding sites that may link to peptide groups that are present on the selective phage 14. Multiple types of PCT and PL compounds 12, 13 may be combined on a selective phage 14 to be carried to the target locations. Large numbers of these PCT and/or PL compounds 12, 13 may be attached to each phage (e.g., hundreds or over a thousand molecules). The quantity of molecules allowed to attach to the nominal selective phage 14 may not, in one embodiment, unacceptably reduce the probability of the phage 14 attaching to the targeted cells, pathogens, or other targeted material 24 below a point of being useful. It should be understood that refinement and removal of most of the improperly linked compounds may be desirable to maximize effectiveness of the treatment or analysis. Bonded PCT or PL compounds 12, 13 that are not bound to phage 14 may be washed from the selective phage 14 and separated by various filtering and separation processes generally known to those skilled in the art. Numerous test cell, pathogen, or material types may be used during the preparations to broaden the target selectivity and/or reduce the probability of attachment to non-targeted materials. All or part of this selectivity process may be repeated as many times as desired to further increase the selectivity. Repeating may, for example, continue until the additional gains provide sufficiently diminished improvements. It is possible to also place a specific non hazardous target material in the body away from a light source to be used to remove phage more quickly from the body, if desired. Furthermore, it should be understood that the selective compounds 10, 20, 30, 40 having the bonded phage 14 may have a modified activating wavelength spectra when compared with the PCT or PL compounds 12, 13 without the phage 14.

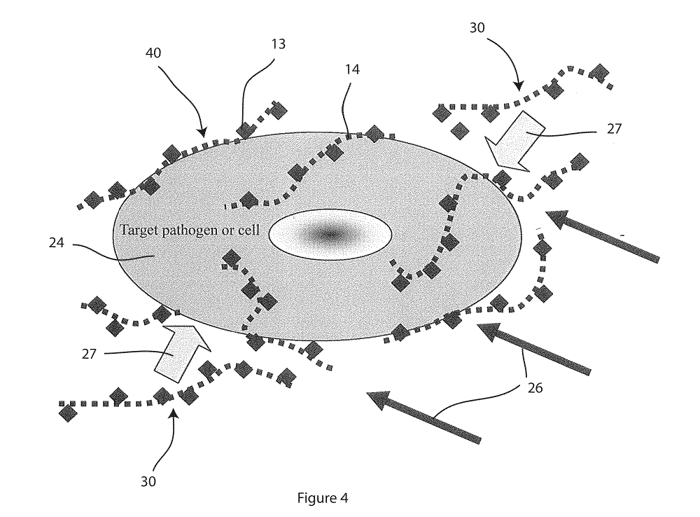

FIG. 4 shows a representation of a target cell 24 with a plurality of the selective PCT compounds 40 attached to the cell 24 and additional selective PCT compounds 40 in the vicinity that are likely to attach to the cell 24 (fat arrows show direction of movement of unattached phage). It should be understood that in other embodiments the selective PCT compounds 40 may instead be the selective compounds 10, 20, 30. The selective phages 14 are shown typically carrying one or more of the PCT compounds 13. Again, the number of PCT molecules per phage may be large. PL molecules 12 may also be attached to the selective phage 14, but are not shown in this example. A combination of the selective compounds 10, 20, 30, 40 may also be used. Incoming light 26 activates the PCT molecules 13 attached to the phage 14. Light photoemission from the PCT molecules 13 (for PCT molecules that are photoluminescent) may be sensed and used to determine if and where the phage are concentrated. This aspect is not shown in this example diagram but it should be understood that such a benefit is anticipated for many PCT compounds 13. Furthermore, the selective PCT molecules 30 that have yet to attach to the target cell 24 may move in the direction 27 toward the target cell 24, as shown in the Figure.

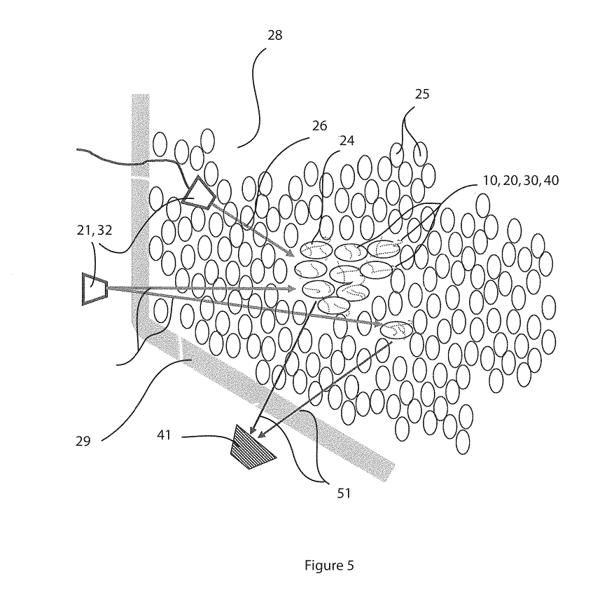

Referring now to FIG. 5, another embodiment is shown. In this embodiment, at least one of the selective compounds using carriers such as monoclonal antibodies or phage 10, 20, 30, 40 is shown after having been injected or otherwise introduced into a body 28 past a skin or tissue surface 29. The injection may be in tissue, fluids, or fat in or near the target cells 24. Injection in many locations that permit the compounds 10, 20, 30, 40 to enter into the blood stream may be acceptable (most likely using a drip intravenous or other injection process). Particularly, the injection may be upstream from the blood supply to the target cells 24 more quickly with minimal filtering of the phage 14 by the body, but the best procedure for introduction of the P1 and/or PCT compounds 12, 13 bound to phages 14 may be determined by the overall objective, location, size, type of target cell, and the type of PCT compound 13 that is being used. Application for surface and wound related pathogens may be made topically on a wound in a solid source, semi-solid or gel, liquid, or even as a gas based spray of the selective phage 14 with PCT and/or PL compounds 12, 13 on the suspected infected area. Membrane transports, ultrasonic energy, heat, and other mechanisms may be used to accelerate the transport of the monoclonal antibodies, phage, or other carriers 14 to the target sites. Once the selective PCT compound 10, 20, 30, 40 is injected or applied, it may take several minutes or up to several hours for the selective PCT and/or PL compound 10, 20, 30, 40 to connect with and attach to the target cells 24 in sufficient concentration for photocytotoxic therapy or analysis. Once attachment is achieved, a PCT activating light source 21 may be used to direct the PCT activating light 15, having a specific wavelength or spectrum, in the vicinity of the target cells 24 having the selective phage 14 bonded with the PCT compound 13 and/or PL compounds 12 attached. The PCT compound 13 releases an oxygen singlet, thereby selectively destroying the target cells or other pathogens and targeted materials 24 with minimal risk to the nearby normal cells 15 that may be only several cell widths away. In this manner, a cell level specificity is used as a safeguard and permits a fast and aggressive treatment for cell destruction with less risk of affecting other parts of the body or destruction of the normal cells 25 that are close to the target cells 24 than in minimally selective light activated photocytotoxic treatments. Because the PCT chemicals 13 only exhibit toxicity during and for a very short time after exposure to a specific spectrum of light and effectively only have toxic properties where and when the activating light is provided, there is little risk to other parts of the body (such as the liver and kidneys) where the phage and photocytotoxins are removed and might otherwise accumulate until eliminated by the body. If non-photoactive drugs were transposed in this way, these drugs may be concentrated in the liver and kidneys and may potentially cause damage, resulting on lower doses and a lower probability of providing a rapid cure.

To prevent over-exposure of healthy tissue between the light source and the cancer or pathogen to reactive oxygen species such as singlet oxygen or super oxide anions, it is preferred that the photodynamic compound possess at least one of limited photostability and photo-oxidation stability. Without this, the higher light absorption could generate a far higher amount of reactive oxygen species near the light source per molecule of photosensitizer.

The embodiments disclosed herein may thus solve one of the most difficult issues for cancer and pathogen therapy: How to direct a highly reactive drug to the correct place in the body, and make it active without significant risk to other parts of the body, especially the liver and kidneys? This novel methodology permits even isolated target cells or pathogens to be identified.

The PCT activating light source 21 may be a light emitting diode (LED), laser diode, gas laser, dye laser, xenon lamp or other light source, and may be arranged in almost any configuration, depth in the body, and level of intensity. For example, the activating light source 21 may be arranged on cables or wires. Access to a patient's deep internal target cells, such as tumors, for illumination by the PCT activating light 15 may be achieved with needle-sized probes using optical fibers or LED's mounted on the probes to deliver the activation light to the desired location(s). If fiber optics are used, the fiber optics may be single or multiple grouping fibers. The fibers may be of many shapes including flexible or rigid ribbons or rods. Fiber optics may also be used to concentrate light from many LED or LED laser light sources into a number of various fibers to provide multidirectional light entry points or multiple points of insertion in the body, or there may be more than one light source used. The end tip of the fibers may be used to emit light along a line, or in any number of spectrum, directions, and/or configurations. The PL material 12 or particle concentration in the fiber, or reflectors may be used to emit light along a flexible or ridged pathway only in controlled directions. If fiber optics are to be inserted into the body or tissue, portions of the fibers other than the light emitting tips may be of a low index of refraction film coated and or jacketed so as to minimize risk of exposure along the fiber due to possible coating defects. Multiple cuts or partial cuts, melted slight index change zones, indentations, imbedded scattering particles, fiber coating imperfections, PL materials, or other structural deviations in the tip portion of the fiber may be used to scatter light in almost any desired light distribution from the tip. Also, any combination of the above may be used. The tip may be a short or long fiber (with or without light scattering mechanisms), wedges, ribbons, lens(es)-on-fiber, canconcentric layered cylinders, and other configurations that brings the desired light spectrum to the surface of the body or into the body at an adequate intensity. The higher the concentration of PL and PCT materials 12, 13 there are at the target site, the less light intensity may be required. Such light sources are considered novel in themselves for these applications and may be used with or without selective phages 14 and selective compounds 10, 20, 30, 40. High intensity, tissue penetrating red and near-visible infrared light (e.g., 665 nm red light with ˜10 nm bandwidth for chlorine e6 Talaporfin derivatives is effective or 780 nm+/−30 nm for ICG) may be used even without breaking the skin for shallow depth target cells, such as certain types of lung, colon, esophagus, eye or skin cancers, if this light spectrum or wavelength is appropriate for activating the PCT compound 13. The entire procedure and arrangement of the activating light source 21 may be optimized by a physician for almost any cell specific treatments in precisely controlled and localized areas. Single or multiple LED's may be used in any number of arrangements. The wavelengths of the activating light source 21 should of course be selected to appropriately activate the photodynamic compounds, or using a spectrum 18 to activate PL compounds 12 that may be used to activate other PL compounds 12 (e.g., a photoluminescent section of a fiber optic inserted, pointed at, or laid on the body).

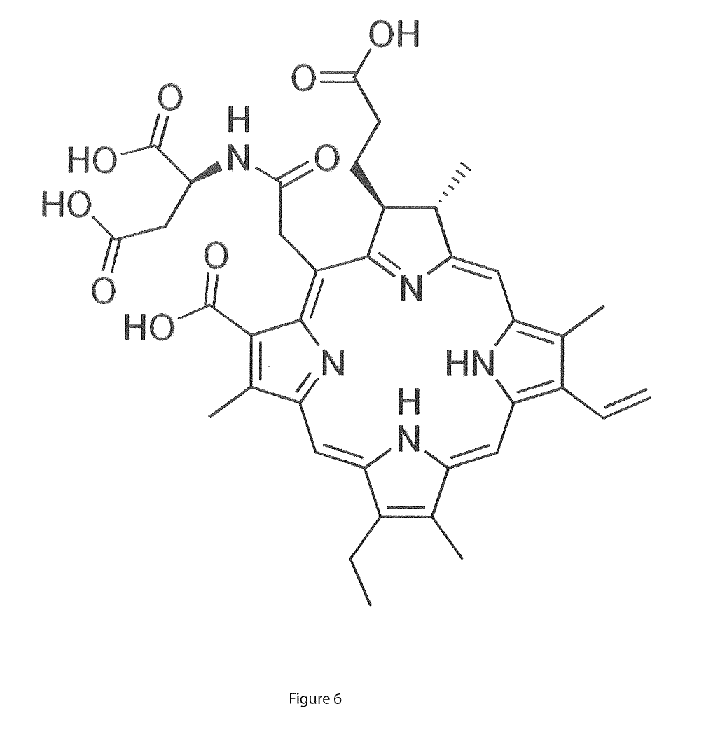

It should be understood that the embodiments described herein contemplate many examples of appropriate PCT compounds 13 that may be bonded to the carriers such as the bacteriophage 14. One example of an appropriate PCT compound 13 is ICG, talaporfin, or chlorine e6. After activation by light having a specific spectrum, talaporfin sodium forms an extended high energy conformational state that generates singlet oxygen, resulting in rapid cell membrane or internal cell damage and initiate cell death. Damaging the targeted pathogens or cancer cells can also be used to activate the immune system to help the body identify and attack the targeted pathogens or cancer cells. Some compounds may photogenerate free radicals such as superoxide anions to induce localized chemical reactivity (e.g., ICG can generate both super oxide anions and singlet oxygen), while others may directly react with a cell or decompose into reactive component(s). The molecular structure of talaporfin sodium is shown in FIG. 6. The chemical structure name of an example compound is mono-L-aspartyl chlorine e6. It has a peak absorption wavelength of 664 nm and releases a single oxygen when activated. Talaporfin is an agent consisting of a derivative of chlorine e6, derived from chlorophyll, and L-aspartic acid with photosensitizing activity. Talaporfin contains four carboxyl groups, three of which are generally available for bonding with the bacteriophage 14. After reaction with a peptide, a conjugate of talaporfin sodium with three peptide molecules or a mixture of conjugates with a different number of peptide molecules may form. This viral conjugate of talaporfin sodium or other PCT or PL materials may have slightly different spectral properties than the unconjugated material(s). Aminolevulinic acid, protoporhyrin IX, several pthalocyanines, and other PCT compounds may be used to selectively target the cells, pathogens, or other target materials. It should be noted that talaporfin sodium contains a vinylene group which also may be used for peptide bonding. However, it should again be understood that talaporfin sodium is only one example of an appropriate PCT compound 13. Another example is profimer sodium. The P1 and PCT compounds 12, 13 should be provided with activated carboxyl groups that may allow their conjugation with N-terminal amino the groups of copies of major coat proteins and their K6 (lysine) aminogroups or other methods of attachment to the bacteriophages. It is also feasible to put up to five copies of the compounds through their coupling with Cys or selenoCys groups of the modified coat proteins pIII at the end of the phage particles. Any compound that has cell destructing properties, or activates or releases a cell damaging element or compound when exposed to a certain wavelength of light may be an appropriate PCT compound 13 in this embodiment. Any variation of this methodology that stimulates a reactive process or immunological effect upon photo excitation using selective bacteriophages to target surfaces in or on the body are contemplated. This may include nucleation for cell growth, removal or detection of other materials such as arthritic nodules, arterial and stint plaque removal, use as local anti-venom (such as bacteria contained in pathogen based venom), fat cell removal (partial and/or shaped removal of fat cells in selected regions by low dose of PCT carrying phage, lower PCT content per phage, and/or by varying the light exposure dose(s)), and removal of other selected cell types or biomaterials (may also be used for partial removal by adjusting the drug concentration time or light dose), and other processes. All treatments may be light activated to localize the effects, but the area exposed to light may be made to be large such as most of the lungs, sinuses, stomach, intestines, lymph nodes, or skin as determined necessary. The majority the body may be trained to remove a targeted cell type such as a cancer, pathogen, or other targeted material 24 as long as the untargeted sensitive places where the phage 14 and PCT materials 13 are concentrated such as the liver or kidneys are not exposed while the PCT 13 is present. Probes inserted into the body may be used to reach areas difficult to expose to light. Injections may be used to deliver the PCT carrying phage and/or PL compounds to difficult to reach areas such as the brain. For example, the liver and kidneys may be treated and may require care being taken to only expose a portion of the organ at a time and not damage too large an area. Care may also be taken regarding immune reactions to verity potential reactions are not overly severe. The treatment of critical areas of an organism where there are barriers to blood (e.g., arterial walls) or body fluids that may be undesirably released should be considered with care so as not to overly weaken such membranes before taking precautions and/or utilizing multiple step treatments. The technique may be used without a PCT compound 13 and be simply a PL compound 12 linked to selective phages 14 for diagnosis or imaging. Other PCT compounds 12 such as profimer sodium may be reacted with a peptide and bonded with the bacteriophage 14 in generally the same manner as talaporfin sodium, as described hereinabove. It should also be generally understood that individual molecules of the PCT compound 13 may also be linked in groups to other individual molecules of the PCT compound 13. This may provide for multiple wavelength and multiple photoreactive properties.

The selective phages 14 or the PL and/or PCT-phage linked compounds 10, 20, 30, 40 may be produced and stored in large batches for future use. Selective phages 14 for many pathogens may be created, stored and linked to the PCT or PL compounds 12, 13 as needed, or linked ahead and stored. Uses of these selective phage 14 linked PL and/or PCT materials 12, 13 may be for diagnosis, locating, and/or treatment in the various examples in this section. This may be advantageous when the target pathogen or cell 24 to which the particular selective phage and PCT compound 10 is meant to attach to is a previously evaluated type of pathogen to which patients may need treatment. For example, this may be appropriate if the target cell 24 is a pathogen such as bacteria, fungus, protozoa, amoeba, or even parasitic organisms such as various types of worms. In this case, large batches of the selective phages 14 or phage-linked compounds 10, 20, 30, 40 for each of several strains or types of pathogens may prepared, mixed, and stored for future use. These selective phage linked compounds 10, 20, 30, 40 may be even more temperature and time stable than current antibiotics which are used to treat similar pathogens. It is also possible to use phages 14 with broader selectivity to broad strains of pathogens. Alternately, multiple individually selective compounds 10, 20, 30, 40 may be stored together to create a hybrid compound mixture that contains selective compounds 10, 20, 30, 40 which may attach to several type target pathogens. For example, all of the individual selective PCT compounds 20, 30, 40 that treat corresponding individual pathogen types and strains may be mixed to create a hybrid compound mixture for broad spectrum treatments or diagnosis. This hybrid compound mixture may be used in the manner described above to treat this broad range of pathogens.

Referring still to FIG. 5, another embodiment may be understood utilizing the selective PL compound 10 instead of the selective PCT compound 40. The selective phage PL compound 10 is shown after having been introduced into a body 28 in addition to the selective PCT compound 40. The selective PL compound 10 (the PL is selective when attached to a selective phage 14) and the selective phage PCT compound 40 (the PCT is selective when attached to a selective phage) may be introduced at any point relative to the other. The selective phage PL compound 10 is shown attached to target cells 24. Attachment of the selective PL compound 10 occurs in generally the same manner as the attachment of the selective PCT compound 40 described hereinabove. Once attached, the selective PL compound 10 may be activated by a PL activating light source 32 which introduces the PL activating light 17 to the selective PL compound 10, as shown. The selective PL compound 10 absorbs the PL activating light 17 and then emits an emission light 15 having a different wavelength. In this embodiment, the selective PCT compound 30 is then activated with light having a wavelength of the emission light 15, rather than with light having a wavelength of the PL activating light 17. Thus, this embodiment utilizes a second level of protection so that only selective PCT compound 30 that is attached to the targeted cells 24 is activated. This helps prevent activation of a small minority (i.e. 1 out of every 300) of the PCT compounds 13 that may not have attached to the targeted cells 24, but instead is located in the vicinity of the normal cells 25.

Another embodiment utilizes the selective PL compound 10 with the PCT compound 12, rather than the selective PCT compound 40. In this embodiment, only the selective PL compound 10 attaches to target cells by way of transport on selective phage. When the inserted or external activating light source induces photoluminescence to the selective PL compound 10, the selective PL compound 10 again emits the emission light 15 which acts to primarily activate the PCT compound 13 that is in the vicinity of the PL emission light 15 rather than the PCT compound 13 that is located further from the emission of the emission light 15. Thus, in this embodiment the PCT compound 13 is not required to be “selective,” or bonded with a carrier such as a bacteriophage 14, in order to achieve selective destruction of targeted cells. Higher intensity light sources are required for this implementation than for the PCT compound 13 bonded to the selective phage 14 embodiment.

Alternately, a single selective hybrid compound 20, 30 as shown in FIGS. 1(b) and (c) containing a PCT compound 13, a PL compound 12, and a carrier such as bacteriophage or other selective transport means 14 is contemplated. Two, three, or more of these materials may be chemically bound together in this embodiment in any possible combination and in any quantities that still permit the phage 14 to be acceptably selective to the target cells, pathogens, or other target materials 24. In this manner, the PL compound 12 may be directly bonded to the bacteriophage 14 and directly bonded to the PCT compound 13. Alternately, the PCT compound 13 may be directly bonded to the PL compound 12 as shown in FIG. 1(c). Thus, the selective hybrid compound 20, 30 may attach to a targeted cell, pathogens, or other targeted materials 24 in the manner described hereinabove with respect to the carrier such as a bacteriophage 14, absorb light of a certain wavelength 17, and emit light of certain wavelength 15, as described hereinabove with respect to the PL compound 12, and react when introduced to light 15 of a certain wavelength as described hereinabove with respect to the PCT compound 13.

Referring now to FIG. 7, multiple different selective or non-selective PL compounds as nanoparticles or molecules 33, 34 may be utilized in any of the embodiments described herein. The normal cells 25 are shown in the region near the target cells 24. For example, different types of carrier such as bacteriophages 14 for each of the PL compounds 33, 34 may be utilized in one procedure or treatment. This may provide additional target cell specificity or broaden the ability for the diagnosis, analysis, or treatment to affect a broader range of target cells. Furthermore, additional PL compounds 33, 34, with or without bonded bacteriophages or other tags, may be used to modify the emission spectrum of the primary selective PL and/or PCT compound 10, 20, 30, 40. For example, a “step” technique may be used to shift the spectrum of wavelengths over a larger range between a first PL activating light 35 and a PCT activating light 36. In this case, a first PL compound 33 is activated by a first light 35 having a first wavelength. The first PL compound 33 emits a second light 37 having a second wavelength. The second light 37 is configured to activate the second primary selective PL compound 34. The second primary selective PL compound 34 emits a third PCT activating light 36 having, a third wavelength which activates the PCT compound 13 attached to a selective phage 14 on the target of interest 24. This allows the activation wavelengths 36 of the PCT compound and the first light 35, which comes from a light source 38 such as an LED, to be very different. The light source 38 may also be the PL activating light source 32 or the PCT activating light source 21. Also, different PCT compounds 13 with different activation wavelengths may be used this way. Again, the PCT compound 13 and each of the PL compounds 33, 34 may be combined on a single selective phage to minimize wasted light. This permits more choices of PL compounds and may be used to prevent unwanted direct activation of the PCT compound 13 from the first light 35 as long as the PCT compound 13 has little activation sensitivity outside of its narrow spectral band, or light outside the PCT activation spectrum range is locally attenuated. Further, since the bonded bacteriophages 14 may affect the spectral characteristics of the PL compound, the “step” process may also allow for a wider range of bacteriophages 14 and multiple PCT materials to be implemented through localized re-emission or absorption of various light spectra.

Furthermore, PL and non-PL dyes may also be used to additionally restrict the penetration of light in tissue or body fluids to nearby potentially sensitive areas, permitting higher luminous flux to be used in the region of the target cells. Absorbing materials such as dyes and organic compounds containing dyes may be used to absorb light to further limit the distance light used in the procedure may travel and thereby provide another control method to shape the light exposure treatment region. Light absorbing-only compounds may also be attached to phages. These light absorbing materials may absorb the activating wavelength, the source wavelength, and any other intermediate wavelengths that are part of the procedure. If attached to normal cells near a photodynamic treatment zone, selective phages with absorbing dyes may reduce the effective intensity range of the activating wavelengths. This technique may be useful when treating liver or kidney cancers with photocytotoxic methods.

It should be understood that there may be many examples of appropriate PL and light absorbing dye compounds 12 that may be bonded to the bacteriophage 14 as contemplated. One example of an appropriate PL compound 12 is K8-1357 produced by SETA Biomedical LLC. This is a squaraine compound, having a low mass, several adjustable spectral characteristics, stable and is attachable to many other compounds. PL compound material classes such as polymethines, porphyrins, rotaxanes, or phthalocyanines may also be appropriate, among others.

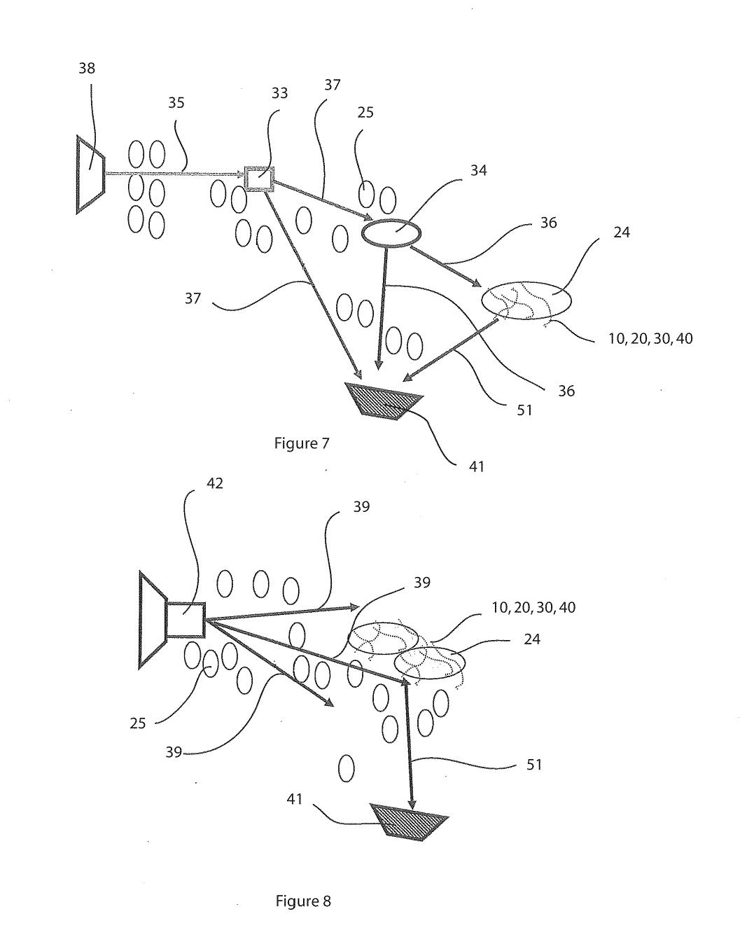

Another embodiment is shown in FIG. 8. In this embodiment, one or more selective PL compounds 10 may be used for imaging purposes in order to detect the size, shape, location, and other physical qualities of target cells with a concentration of selective phage 14. This selective PL compound 10 may be used with the PCT compounds 13. Further, if the PCT compounds 13 are sufficiently photoluminescent and concentrated, the selective PCT compound 40 or other PCT compound combination may then be used in place of the PL compounds 12. In the embodiment shown in FIG. 8, a PL activating light 17 is scanned, using a PL and/or PCT activating light scanning source 42, over a target area having the target cells 24 to which the selective phages with PL compound(s) and/or PCT compound(s) 10, 20, 30, 40 are attached. The selective phage 14 attached to the PL compound 12, for example, may have substantially similar properties as the selective PL compound in FIGS. 1(a) -1(c). However, it should be understood that any particular or individual selective PL compound 10, 20, 30 referred to herein may utilize different PL compounds 12, bacteriophage strands 14, or other different molecular properties. In general, PL compounds 12 typically emit an emission light 15 having a longer wavelength than the light emitted by the activating light 17. These longer wavelength emissions may be detected and used for imaging tumors, localized infections, selected cells and other targeted materials. The spectrum, polarization, and coherence may all be measured. Transient time based changes in these parameters may be observed using high speed spectrometers and optical sensor arrays to gain additional information and permit larger numbers of PL tags to be simultaneously used. Larger number of PL tags 12 attached to phages 14 selective to different pathogens, cell types, or target materials 24 permit greater analysis and diagnosis. The detector may pick up light through a fiber optic or bundle for small spots. The detector may capture emitted light from a larger area or be used directly if small enough. This applies to all light detectors contemplated.

In one example of this embodiment still using FIG. 8, broad or narrow spectrum light 39 using yellow to near IR wavelengths is line or raster scanned with a line raster scanner source 42 over the area potentially containing the selective phages 14 with PCT and/or PL compound(s) 12, 13 and then a spectrum sensitive detector 41 provides an output spectrum signal as well as intensity spectrum. The scanning light spectrum may at a minimum contain a range of the excitation or activation wavelengths of the planned selective PCT and/or PL compounds 12, 13 to be used. The detector signals are synchronized with the line raster scanner 42 to create a depth image of the selective phage linked PCT and/or PL compound 10, 20, 30, 40 concentrations and other light from the scanned 42 that is reflected or scattered. The type of tissue or fluids will affect the ratios of the penetration depth, but this may be reasonably predicted. This process may also be performed with a scanner that scans a monochromatic beam of light, but changes the wavelength during the scanning process and synchronizes this with the detector.

A simple probe consisting of one or more each of sensors and light sources may be simply moved over or into the body by hand to detect where and if a concentration of PL tagged or PCT attached to selecting phage may have occurred after the subject has been provided with the selective phage. Such a system may locate and determine the type of pathogen present when used on a subject treated with a prepared selective PL tagged materials, or PCT compound(s). The light source may provide a single wavelength light, broad spectrum light, laser light, or scan multiple wavelengths. The sensor may pick up a broad bank, specific spectrum if looking for a particular spectrum or a small grouping of spectrum. The light source and sensor may be at the scanner, or may be remote from the scanner through fiber optics (a remote light source through fiber optics may frequently be the most practical and versatile approach and the fiber optic or fiber bundle may then potentially be flexible and disposable). Detection of coherence, polarization, reflected, absorbed, and emitted light are all of potential use in analysis, along with the option to provide intense light at the PCT activation wavelength if a photo-activated PCT therapy is desired. Scanning the wavelength and polarization of the incoming light, plus analyzing the spectrum, polarization, and coherence of the detected light may be the most powerful use. However, simple diagnosis or treatment may be done with a simple LED or laser diode spectrum source. A simple photocell detector with one or more filters may be designed using available methods to those skilled in the art. Hand scanning the light source and detector together or independently (with an optional gel or liquid such as glycerine) and suitable electronics may also be designed using available methods to those skilled in the art.

In another example of this embodiment, again shown in FIG. 8, the narrow spectrum light 39 is line or raster scanned with a line raster scanner source 42 over the area potentially containing the selective phage attached to PCT and/or PL compound(s) 12, 13 repeatedly, after which a simple broad sensitive detector 41 placed on the skin or in the body may provide an output spectrum as well as emitted or reflected light intensity spectrum. The detector may pick up light through a fiber optic, area light concentrator light guide or directly from the skin surface. The scanning light spectrum range may at a minimum contain a range of the excitation or activation wavelengths of the planned selective phage linked PCT and/or PL compounds 10, 20, 30, 40 to be used. The detector signals may be synchronized with the line raster scanner 42 to create a depth image of the selective phage linked PCT and/or PL compound 10, 20, 30, 40 concentrations and other light from the scanner 42 that is reflected or scattered. The type tissue or fluids will affect the ratios of the penetration depth, but this may be reasonably predicted. It is also possible to use both a narrow spectrum light with a broad spectrum light at the same time. Furthermore, the light scanners or the detectors may be inside or outside of the body of the target cells being scanned. Scanning in this embodiment may be done quickly because the PL compounds may have response times of much less than 1 microsecond, once activated by the activating light source. This technique may also be used to form an image. Fourier transforms of rapidly changing wavelengths and/or intensity, Fluorescence Correlation Spectroscopy, Fluorescence Lifetime Imaging (FLIM), and other methods to optimize the information obtained during scanning may improve image quality.

In this embodiment, the wavelength of the scanning activating light 39 may be varied in a repetitive manner to provide depth imaging. Depth imaging utilizes the fact that longer wavelengths penetrate tissue farther than shorter wavelengths. This may be calibrated to certain types of tissue to provide depth estimates. This may provide physicians fast and real time identification of the size and location of multiple tumors, infections, or other of the targeted areas in the body. Multiple different selective phage linked PCT and/or PL compounds 10, 20, 30, 40 may be used to emit multiple emission light spectrum having different wavelengths for additional information during scanning. It is also possible to scan yellow, orange, red, near infrared spectrum, and even infrared light to detect dye absorber tags, providing both absorbers and emitters to increase the number of possible analysis of diagnosis tags for a large mixed grouping for various selective phages, and through these tagged selective phages, identify which pathogens may be present when using a mix of pre-selected and tagged phages each selective to a specific strain or class of pathogen. It is also possible to activate the PCT compound 13 or the selective PCT compound 10 during imaging by using appropriate wavelengths and PL compounds 12. It is also to possible purposefully not activate the PCT compound 13 or the selective phage 14 linked PCT compound 13 during imaging if the appropriate wavelengths and PL compounds 12 are used in this procedure. This may be most easily be done by scanning light with longer wavelengths than the activation wavelengths 15 of the PCT compound 13.

A plurality of the selective phage attached to PCT and/or PL compounds 10, 20, 30, 40 may be used, wherein each individual of the selective PCT and/or PL compounds 10, 20, 30, 40 may be configured to attach to a particular of bacteria, fungus, other pathogen, or other target material. This multiplicity may be combined on one selective phage, or multiple selective phages. Each individual of the selective phage linked PCT and/or PL compounds 10, 20, 30, 40 may be configured to emit a different colored light, thereby allowing matching of the particular bacteria, fungus or other pathogen with an individualized color.

Furthermore, in this embodiment a detector 41 is used to detect the emitted emission lights 51. The detector 41 may be placed further away from the targeted area being imaged or analyzed, providing more flexibility and a better chance of not needing to enter tissue with a detector probe (not shown). This also provides a higher contrast image with less scattering. Many commercial visible to IR wavelength range light detectors (spectrometers) exist that may read the intensities vs. wavelength of the various PL compound emission or non-PL dye absorptions quickly and plot selected emitted or reflected wavelengths of light. Due to light scattering in tissue, processing of the time and wavelength information that is detected is required to for basic images and/or pathogen type analysis or diagnosis. Fiber optics, light guides, and/or light concentrators may also be used for the detector end. The end of the fiber optic may be flared or split to permit accumulation of a large amount of emitted and reflected light. Multiple detectors 41 and PL and/or PCT activating light sources having different locations may be used to gather more, or different, imaging data.

It is contemplated that imaging with the selective PL compounds 10 in this manner may be utilized in fields such as forensics. For example, if you spray and wash an area with the selective PL compound 10 that is targeted to the cell type that is being detected, very small samples of specific individual cells may be found mixed other untargeted cells.

Ultrasonic energy has been shown to improve the effectiveness of light activated treatments with Talaporfin Sodium and may also provide additional useful imaging or diagnostic information. The benefits of utilizing ultrasonic energy are likely to also apply to this Selective-PCT (SPCT) therapy.

It should be understood that the herein described embodiments may be applied to any cell or material that a bacteriophage or other tag may attach to. Examples include cancer cells, pathogen treatments, removal of non-cancerous cells, benign tumors, fat cells and cosmetic surgery, fungus or bacteria and potentially the removal of non-cell materials that might be affected by a photoreactive element that may be bonded to a phage such as arterial plaque. The carriers such as monoclonal antibodies, bacteriophages, or other carriers and reporting tags 14 may be configured to attach to almost any cell, and some non-cell materials, depending on the individual strain of the bacteriophage virus or other tag used. For example, the bacteriophages or other tags 14 may link to reactive NHS esters which may be bound to amino groups (NH2) of biological molecules, and maleimides which may be bound to thiol (SH) groups, and many other groups. These bacteriophages or other tags 14 may be bound to oligonucleotides or antibodies providing further selective binding to complimentary oligos or antigens. Non-covalent bonds may form between one of the bacteriophages or other tags 14 and large molecular weight biomolecules such as proteins, lipids, membranes, cells and many other forms of biological matter. Photodynamic compounds may be used for the binding to proteins (e.g. BSA, HAS), immunoglobulins (IgG), oligos, peptide, avidin, biotin, enzymes, along with many other high and low-molecular weight compounds. Furthermore, chemicals produced by target cells may also be targeted by the bacteriophages or other tags 14. For example, enzymes, peptides, proteins, and other compounds produced by cells or pathogens may be targeted by the bacteriophages or other tags 14. It is also possible to target the bacteriophages or other tags 14 to chemicals associated with blood vessel creation, cell division, or other processes that are more frequently associated with aggressive cancer and or pathogen cells than normal cells.

Thus, many currently ‘incurable’ cancers may be effectively treated. The treatment described may be an outpatient or short hospital stay procedure when the affliction is near the surface of the body, even when the affliction is over relatively large areas. Once the process becomes routine, treatments may frequently be office visits potentially lasting about an hour within a few days after a biopsy and after the selective phage attached to the PCT have been prepared. Unlike cancer treatments that may be fully individualized, treatments for known pathogens may be prepared far ahead for most patients and this treatment may address most antibiotic resistant strains of bacteria. The therapy and analysis approach may potentially be a minimally invasive or even non-surgical technique, depending on where the tumors, target cells, or pathogens are located. The light sensitive chemical part of the process may decompose harmlessly after a few hours or days of time depending on the PCT material used, removing future sensitivity to even the specific chemical trigger wavelengths. The short-lived characteristics of the example singlet oxygen (typically active about a nanometer or less distance from the activated PCT material) in potential selective PCT compound treatments and the nanosecond range response time of many PL materials keep the chemical activity of these PCT materials localized at the exposure sight. It is also possible to use PL phosphorescent compounds having much slower responding materials, however. Additionally, embodiments may be applied to animals, plants or any other living creature in addition to humans. Embodiments may also have applications outside of a living body, such as the forensics example described hereinabove.

Filters (dichroic, diffractive grids, pigments, or dye based), light amplifiers, photonic crystals, photoluminescent (PL) materials, electrochromic switches, Nanoquantum's® CPC materials, light guides, fiber optics, light diffusers, micromirror and larger size mirror, lens or mechanical scanners, light scanners or other instruments may be used to narrow or alter the spectrum or redirect the light from light sources such as lasers, light emitting diodes (LEDs), Organic LEDs (OLEDS), inorganic electroluminescent devices, metal vapor lamps, arc lamps, fluorescent lamps, and many other type light sources to provide a suitable wavelength spectrum to the PL and/or the PCT. molecular compound(s) (including nanoparticles made from of these materials. These spectral altering, light redirecting, and light generation instruments may be used to provide light as needed to optimize the selective activation of materials or compounds in the processes described herein.

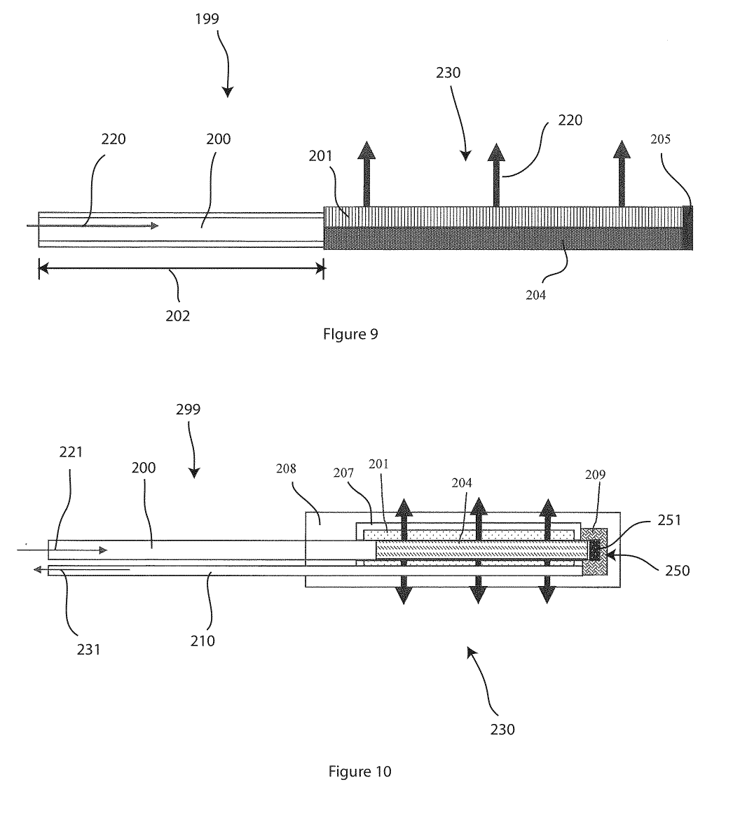

Several LEDs that emit at the desired wavelength may be wired or otherwise electrically linked together to provide a thin long light source probe, as is currently practiced for conventional light activated therapy. These type light probes have heat issues in the body at high luminance and are often expensive, but are reasonably effective. One embodiment of an improved light source probe 199 for photodynamic therapy is shown in FIG. 9. The light source probe 199 may be a light altering instrument. The light source probe 199 may further be a spectrum converting filter combined with light reflector and/or scattering material. Such a filter altering instrument may also be used in a transmissive mode or a reflective mode, depending on what is best for the specific situation. A filter, using a photoluminescent spectral altering instrument, may be placed by the light source 199 or at the end of a fiber optic bundle or optical fiber 200. The filter may also be placed elsewhere between the light sources and an area to be radiated with light. It should be understood that the word “fiber” that is used in a tip section portion 230 of the device includes all of the primarily horizontal light pathways such as single or multifiber, ribbon, tubes and coatings, fiber bundles, solid and gel or liquid plus solid light guide variations. The tip section 230 is the portion of the device that emits a light 220. Light of a spectrum required to activate the photoluminescent material 201 in the probe is directed along the fiber or ribbon 200 from a high intensity source (e.g., blue light or broad spectrum) LED. The fiber optic or ribbon 200 may have a length 202 up to several meters long as desired, and may be jacketed up close to the point of body contact (not shown). A cover or coating may be placed on the probe of a transparent medically acceptable material that is long enough to cover the end and any part of the cable and probe within several centimeters of the body. The tip section 230, or light altering portion of the instrument 199 may consist of a layer of photoluminescent material 201 over a core optical fiber (e.g., 0.5 mm diameter core coated glass, acrylic or polystyrene fiber with ˜10 nm thick low index of refraction fluoropolymer coating) or the fiber optic bundle 200. The photoluminescent material 201 may be of concentration and thickness to shift as much of the incoming light energy as possible into the desired output light spectrum. This example shows an optional optical reflector underneath the length of the light emitting portion of the probe with cuts, surface roughness, or other scattering mechanism in the base of the fiber. Forms, cuts or other structures may be designed to direct light as desired when used with the other elements of the light altering unit, and minimize unnecessary light trapping. Surface deformities in the fiber and a low index of refraction coating may be formed so as to scatter light evenly along the light emitting part of the probe (which may be several centimeters). A reflective coating 204 on one side may also be used to further direct the light 220. At the end of the fiber 200, an optional reflective cap 205 is shown to redirect light back down the optical fiber to increase the intensity of light out the top.

It is possible to replace the emitting section of the fiber 200 with a specially prepared tip with several novel features. For example, adding scattering material, such as microscopic glass beads, into or embedded through the low index of refraction coating in the tip of the fiber under heat and pressure is contemplated. Also contemplated is replacing the core fiber of the tip with a photoluminescent material doped fiber photoluminescent at near the desired wavelength range (e.g. red) and optionally coated with another layer of higher concentration photoluminescent material to further tighten the emitted wavelength with or without the rear reflector. Further, a fiber bundle, paddle of fibers, or solid light guide may be used to distribute the light emitting area along a line or 2D surface. In the case of a rod, the device may be scanned over tissue inside or outside the body to identify if the PCT or PL carrying phages have concentrated in any region, and expose that region to the activating light. Additionally, a rod or 2D array may be prepared (as shown in FIG. 9) with a 2D array of “pixels” of controlled light. It should be understood that FIG. 9 may be considered a cross section view if viewed as a 2D array. By rastering or scanning the 2D array near various likely search areas, or moving or sliding the linear array over potential target areas, the location and magnitude of concentrations of tagged selective phage near the detector may be identified. Scanning may also be performed in the body with minimally invasive surgery using thin linear and/or flexible probes with optical fibers for optional sensors.

All the above embodiments of the light source may be uniquely constructed with photonic lattice patterns on the outer surface under the protective cover or coating. The photonic lattice pattern may be on top of most of the filterers and PL material. Furthermore, dichroic or grating filters may be on either or both sides of the PL material. These materials may also be in place of the PL material, depending on the objectives of the embodiment. The PL material may be a CPC structure with small fibers or nanofibers.

The uniformity of light emitted along the tip section 230 may be modified based on the thicknesses of coatings, light reflecting and scattering geometries in and along the core fiber, the PL and filter densities and concentrations, and other factors of optical engineering design.

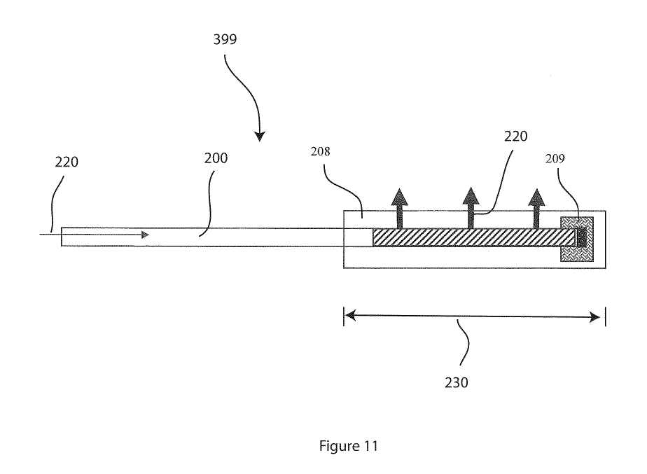

In FIG. 10 a slightly more complex light probe 299 or scanner is shown. This tip section 230 may be used with highly selective photodynamic methodology, and may also be suitable for use with any prior art photodynamic therapy and photocytotoxic therapy methodologies that are less selective. This probe has all of the same elements and options as the probe in FIG. 9, including the fiber or ribbon 200 having the length 202. The probe 299 includes the tip section 230 which contains the same photoluminescent or filter layer 201. The tip section 230 may be encapsulated in a medical compatible protective coating such as polyethylene or PTFE 208. The core may be transparent fiber optic or may be a linear or 2D area light source array with sensor(s) for the emission wavelengths.

FIG. 10 shows the addition of a two part overlapping reflective end cap 250 on the fiber 209. This end cap 250 may minimize lost light at the tip ends with the overlap being at least 50% of the thinnest part of the fiber. It should be understood that the word “fiber” as used in this section includes the primarily horizontal light pathways and includes all of the single or multifiber, ribbon, tubes and coatings, fiber bundles, or light guide variations. This end cap may be a highly reflective metal such as silver or aluminum 209 that may be placed or coated on the end piece 251. This reflector may be processed or made as a single piece. The second part of the cover 209 that extends further over the end of the tip may be a material that absorbs the input light and/or the light emitted from the tip so as to not form a bright spot near the end of the Tip Section 230. If there is a desire for a point light source at the end of the tip section(s) 230, then the end reflectors may not be necessary.

In the embodiment shown in FIG. 10, an optional outside optical filter or photonic lattice array 207 to further narrow the emission spectrum is also shown. This outside optical filter or photonic lattice array 207 may be imprinted onto or into one or more the coatings on the fiber. Imprinting may be performed with early industrial high resolution or e-beam/ion beam based lithographically, interference, conventional light based lithography, mechanical imprinting and/or by other known patterning methods. A sensing fiber 210 is shown that may consist of multiple fibers or an additional higher index of refraction coating around part or all the probe with low index of refraction additional coatings to trap light 231 that will be directed in part back down a optical fiber cable. The optical fiber cable may be, in one embodiment, opaque jacketed. Light emitted from the patient or test may be sensed by a remote optical analysis system. If such sensors are used in the tip section, the high intensity input light to the body may be time-separated from the picked up sensor light by pulses. Additionally, a filter may be used to block the shorter PL and or PCT activation wavelengths such as dichroic filter that may pass only light above the input spectrum to the tissue or other material being analyzed or treated.

Sensors may also be placed in the tip section of the device. For example, sensors consisting of photosensors in the tip with different dichroic or gratings may also be used. A micro fluorescence or other optical sensor electrically connected to the control system may be utilized. The sensor(s) may also be placed further away from the head (either attached to the tip or not) and located near the tip section that provides the activating light.

Multiple tip sections 230 may be used at the same time. The tip sections 230 may be flexible to make it easy to shape them at localize light intensity where needed. Any of the tip Sections in FIGS. 9, 10, and 11 may potentially be wound and shaped or designed as needed. For example, an array or tips of a coil may be used similar to a bandage over a wound on a patient being treated. If analysis of the region being treated is not required, these tips may be very simple. These tip sections 230 may be made disposable to avoid the need for sterilization risk between patients.

FIG. 11 provides a simple embodiment of a potentially flexible tip design 399. In this embodiment, high luminance light 220 of the desired emission wavelength spectrum may be directed along the coated optical fiber 200. The fiber 200 is optically coated and may be jacketed almost to the tip section, if desired. The spectrum is not converted to another spectrum, except by the PCT and PL materials attached to the selective phage. The light 220 is directed out of the fibers in a desired pattern using several novel techniques with common materials. For example, a mechanical or laser may cut at angles part way through the fiber. Further, changing the optical index or refraction of this fiber core in shapes is a novel way to obtain many angled “slices” that do not cut though the fiber using short wavelength lasers and/or high intensity light. For example, PMMA fiber cores and a UV laser may be used to break most of the polymer bonds but not melt the PMMA fiber at 45 degree angles. The PMMA fiber may then be rotated and repeated so as to reflect light out in all directions or in preferred directions. For example, one may first expose 0.5 mm diameter thin fluoropolymer coated fibers to 405 nm scanning lasers of ˜20-100 mw power with a 0.2 mm or smaller diameter laser beam with 0.5-10 second per slice at ˜45 degrees to the fiber. One may scan fast enough as to not over heat the fiber during exposure while reaching a high exposure dose. Next, one may longitudinally separate the slice distance by about 2 mm for a 5 cm total length tip section and then rotate 90 or 120 degrees and repeat the exposure to slightly reflect light outward at each exposure point. Then, one may bake these angled cross section patterned fibers in nitrogen and a 20% hexamethyldisilazane (1,1,1,3,3,3-hexamethyldisilazane; HMDS; OAP; 1,1,1-trimethyl-N-(trimethylsilyl) Silaramine; Bis(Trimethylsilyl)Amine) vapor at 90dec C. for 1 hour. This may result in a series of slightly different index of refraction “slices” within the fiber. At each of these slice points, a small amount of the incoming light may be reflected outward. Next, adjustment may be made to the number of slices, angles, and distance between the slices to obtain the desired uniformity. Furthermore, adjustment may be made with HMDS treatment temperature and time and laser exposure dose to obtain the desired uniformity.

The number of exposure slices and the angles may be varied. The light 220 may be generally directed on one direction. Alternately, by varying the angles and positions, a more random light output may be obtained. Other compounds such as trimethylaluminum may also create such index of refraction change effects along exposed PMMA regions. Small changes in index of refraction may even occur without high temperature chemical vapor. This technique may also work with fibers, ribbons, and sheets of many polymeric materials. Activation of sensitizers in an organic matrix may also potentially have a similar effect after baking of the exposed fibers. The baking steps in a number of silation or organometalic compounds may increase the change in index of refraction at the exposure zones. Alternately, the baking may increase the change in index of refraction at non exposure zones depending on what chemical is used. It should be understood that other compounds may be absorbed to change the refractive index in a select treated or untreated region of the sheet, ribbon, or fibers.

Nicks and dimples of many shapes and sizes on fibers, fiber or ribbon bundles, ribbons, or sheets of materials other light scattering methods may provide scattering, and may be used in conjunction with other methods to carefully extract light out of the fibers in a controlled way. Microscopic sized or very small organic, gas, or glass spheres, bubbles, chips, or full or partially reflective metal may be imbedded in the fiber surface using solvents or heat and pressure to induce scattering of light. Liquid or gas bubbles may also be formed in the fibers. Embedding with heat and pressure may include rolling the fibers in the material under a high enough temperature to soften the fiber, ribbon, or sheet. This process may be used for materials that may soften under heat with minimal decomposition or transparency change. Metal particles and many other transparent materials may work in this manner. The number of sides, size of the particles, coatings, and matted of pressing of fibers, sheets, or ribbons may change the scattering properties of the fibers, ribbons, or sheets.

These techniques were briefly discussed regarding the embodiments shown in FIGS. 9 and 10 and are a novel way to redirect light out of the fibers. The protective coating 208, compatible with medical applications, may cover the finished tip section 230 and at least several centimeters of beyond the portion of the probe 199, 299 that might be used in the body. The highly reflective end cap 209 may prevent light at the fiber end from creating a bright spot (unless desired) and an optional side reflector may be used to direct light out the sides. This may be a two part cap 250, such as the embodiment shown in FIG. 10, with an absorbing coating 209 over the cap extending a short distance from the end 251 of the tip section 230 to minimize formation of a bright spot at the tip end. This method may be used to redirect light in all directions or a preferred direction from the tip section.

As was the case in the embodiments depicted in FIGS. 9 and 10, multiple fibers, fiber bundles in many shapes, waveguides, and other approaches to scatter or redirect light sideways are contemplated. Any of the above embodiments shown in FIGS. 9, 10, and 11 may be modified to work with OLED light sources. The optical cables may be replaced by electrical feed wires and the core fiber may be replaced by an OLED strip or sheet emitter. Any of these arrays or probes in FIGS. 9 and 10 may contain one or more light sensors to detect characteristic light from photoluminescent PCT or PL compounds 12, 13 that have concentrated by the selective phages 14 and areas of interest. Many pixel sites may be turned on or may also be turned full over the target region. Additionally, the light source intensity may be increased for rapid high light intensity treatments. This feedback may also be used as a follow up repeat procedure to determine if the prior treatment was successful and if groupings of cancer cells, pathogens, or other target material 24 were still present. The target exposed area may be automatically determined and the exposure region and depth may be controlled using feedback. This may minimize unnecessary light exposure in surrounding tissue while the PCT is in the body.

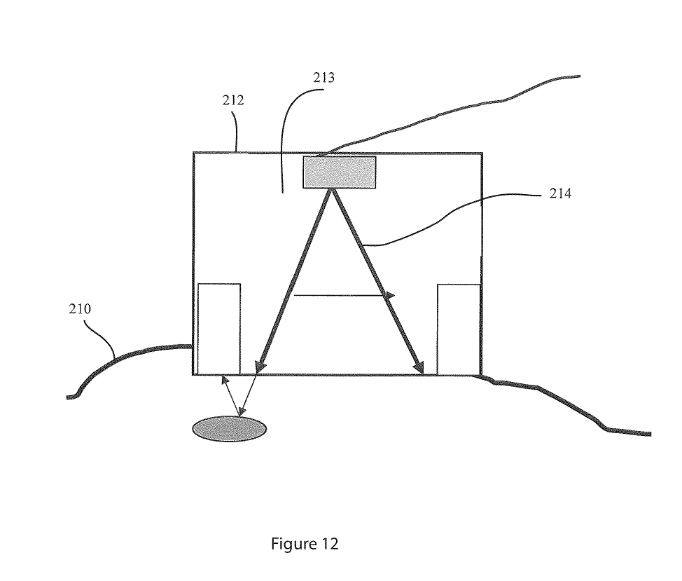

FIG. 12 provides a basic concept view of a scanning apparatus. In this example, a laser or high intensity incoherent light beam 214 is scanned across a tissue surface 210. The scanning process may take place in a cover or box 212 to minimize noise from ambient light reaching the sensor. For example, an optical scanner 213 may be a micromirror scanner that is run electrostatically or with piezoelectric devices for a linear, polar or raster scan.

In this example, light from the scanner and light source system may penetrate the tissue, reaching a targeted area having selective carriers such as phages with photoluminescent PCT and/or PL attachments. The PL attachments absorbing the scanned light may then emit characteristic emission spectra. This emission is then picked up by the optical sensors similar to those previously discussed. The point in the scan corresponding to when the scanning light beam location moves onto and off of the concentration area of the tagged selective phage provides information about the location of the area to be treated. Thus, the high light intensity longer time exposure may be more precisely planned. If there is not a concentration of selective phage, there may not be a need to perform the photocytotoxic treatment unless, for example, it is just a second pass to kill possible remaining isolated cells or pathogens.

For all CPC light sources discussed herein, a LED, xenon lamp, plasma, or laser light source or other suitable source may be provided to power the light source or directly illuminate the skin. Filtering of long wavelength over two micron infra-red light may be desired to reduce heat and/or light at wavelengths of light not being used for the treatment. Light from these sources may be fed into an optical fiber, placed in the body, and/or directed onto the body surface to be treated.

Arrays of light sources may be used to concentrate light in a fiber or over an area. A desired light spectrum may activate one or more of the photocytoxic compounds 13 or the PL compounds 12. If it is desired to use a narrow light spectrum source filters (pigment, dichroic, photonic lattice, interference, etc. . . . ), high concentration PL converters (mixed dyes if necessary) may be used to limit the range of wavelengths.

Scanning mirrors (e.g., piezoelectric or electric field controlled) may be used in conjunction with probes or single point light sources with the scanners and LEDs or laser diodes being located at the end of a probe. These scanners may also be fitted to optical fibers. The fiber optic may further be removable from the light source in any of the above described embodiments. A coupling may be made using a soft or uncured index matching gel in a holder in order to facilitate ease of attachment and detachment. This may permit the fiber optic and probe to be removable.

There may be an optional disconnect point between the fiber from the source and the fiber going to the tip, so that a disposable length may be created. The tip may be a continuation of the same fiber optic, or an attached piece of material. The material may be flexible or ridged. The ridged pieces may be heated and reshaped, or molded to a desired shape. The tip may be a fiber, ribbon, or other shape. The tip may be an optical fiber (coated with higher index of refraction film such as PTFE or silicon nitride) of glass, plastic (e.g., PET, PMMA, or polystyrene), or many other available and reasonably transparent or translucent materials. Cuts or index of refraction discontinuities (hereafter generically called cuts) may be made in the tip material to partly redirect some light outside the fiber at each cut. A different index of refraction translucent or transparent material than that of the fiber may be inserted between the cuts to glue the fiber back together (air may also provide an index change).

The number of cuts, width of the cuts, space between the cuts, and index of refraction of the interim materials may determine what percentage of the input light to the tip light is directed out of the sides of the fiber per unit length. The direction of the cuts and scattering effects at the cuts may mostly determine the direction the light that may leave the fiber. Multiple cuts in different directions may direct the light in multiple directions (e.g., cross cuts) from the fiber, or reflective coatings may be used to help direct light in a general direction.

It is possible to use a PL-dye (organic or inorganic or use optically active tip materials) or a non-PL dye-pigment doped tip material to shift the wavelength spectrum or absorb undesired wavelengths. It is also anticipated that the tip may be all or partially coated with reflectors, PL materials, layered dielectrics, or optical filters or various type to change the output spectrum or light direction.

The cuts may be made by a laser, knife edge or razor blade, hot filament, or other cutting means available that is compatible with the fiber materials. If a laser is used, the index of refraction change from melting the materials may be used to form the partial reflectors. It is also possible to use a collimated light source or laser, electron beam or ion beam to expose a tip material such as PMMA and break the polymer bonds to create a partly reflective boundary. It is also possible to dope the material with a sensitizer and expose this material to form the reflective zones.

An end cap may be used to block light at the end of the tip. If it is reflective to minimize wasting light, a light absorbing coating may be used to prevent undesired lateral reflections and poor uniformity of light intensity near the end cap.

Elements of the embodiments have been introduced with either the articles “a” or “an.” The articles are intended to mean that there are one or more of the elements. The terms “including” and “having” and their derivatives are intended to be inclusive such that there may be additional elements other than the elements listed. The conjunction “or” when used with a list of at least two terms is intended to mean any term or combination of terms. The terms “first” and “second” are used to distinguish elements and are not used to denote a particular order.

While embodiments been described in detail in connection with only a limited number of embodiments, it should be readily understood that the invention is not limited to such disclosed embodiments. Rather, the invention may be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

I claim:

1. A method of selective photodynamic therapy comprising:
introducing a selective photocytotoxic compound to a body having a target cell, wherein the selective photocytotoxic compound is configured to at least one of selectively attach to and selectively enter the target cell; and
activating the selective photocytotoxic compound, at least one of directly and indirectly, with a light source.

2. The method of claim 1, further comprising bonding a photoluminescent compound to the selective photocytotoxic compound.

3. The method of claim 2, further comprising activating the photoluminescent compound with the light source directly such that the photoluminescent compound emits an activating light having a wavelength to activate the photocytotoxic compound.

4. The method of claim 1, wherein the selective photocytotoxic compound includes at least one photocytotoxic compound bonded with a selective carrier selected from the group consisting of monoclonal antibodies, bacteriophage and a polymer.

5. The method of claim 1, wherein the selective photocytotoxic compound, when activated by light, generates at least one reactive oxygen species selected from the group consisting of a superoxide anion radicals and a singlet of oxygen.

6. The method of claim 1, wherein the selective photocytotoxic compound is also a photoluminescent compound with an identifiable characteristic emission spectrum when activated.

7. The method of claim 1, further comprising:
determining one or more bacteriophages that will selectively attach to the target cell;
amplifying the number of bacteriophages; and
bonding a photocytotoxic compound to the determined one or more bacteriophages to create the selective photocytotoxic compound.

8. The method of claim 1, further comprising:
determining one or more monoclonal antibodies that will selectively attach to the target cell;
bonding a photocytotoxic compound to the determined one or more monoclonal antibodies to create the selective photocytotoxic compound.

9. The method of claim 1, further comprising at least one of injecting and diffusing the selective photocytotoxic compound into a body past a tissue surface.

10. The method of claim 1, further comprising attaching a plurality of the selective phtotocytotoxic compound to one of the target cells.

11. The method of claim 1, wherein the phtotocytotoxic compound is selected from the group consisting of a chlorin e6 derivative, a cyanine derivative, a bacteriochlorin derivative, a squaraine derivative, and a phthalocyanine derivative.

12. The method of claim 1, wherein the target cell is at least one of a bacteria, a fungus, a protozoa, an amoeba, a parasitic organism, a cancer cell, fat cell, biofilm, vascular plaque, and a cancer cell.

13. The method of claim 1, wherein the selective photocytotoxic compound possesses at least one of limited photostability and photo-oxidation stability.

14. A method of selective photodynamic therapy comprising:
introducing a selective photoluminescent compound to a body having a target cell, wherein the selective photoluminescent compound is configured to at least one of selectively attach to and enter the target cell;
introducing an activating light to the selective photoluminescent compound, wherein the photoluminescent compound is configured to absorb the activating light and emit an emission light having a different wavelength than the activating light; and
activating a photocytotoxic compound with the emission light of the selective photoluminescent compound.

15. The method of claim 14, further comprising bonding the photocytotoxic compound to the selective photoluminescent compound.

16. The method of claim 14, wherein the selective photoluminescent compound includes at least one photoluminescent compound bonded with a carrier, wherein the carrier is selected from the group consisting of a monoclonal antibody, a polymer and a bacteriophage.

17. The method of claim 14, wherein the photocytotoxic compound generates at least one singlet of oxygen when activated.

18. The method of claim 14, further comprising:
determining one or more bacteriophages that will selectively attach to the target cell;
amplifying the number of bacteriophages; and
bonding a photoluminescent compound to the determined one or more bacteriophages to create the selective photoluminescent compound.

19. The method of claim 14, further comprising:
determining one or more monoclonal antibodies that will selectively attach to the target cell;
bonding a photoluminescent compound to the determined one or more monoclonal antibodies to create the selective photoluminescent compound.

20. The method of claim 14, further comprising:
at least one of injecting and diffusing the selective photoluminescent compound into a body past a tissue surface whereby the selective photoluminescent compound selectively attaches to the target cell; and
injecting the photocytotoxic compound into the body past the tissue surface in the vicinity of the target cell.

21. The method of claim 14, wherein the phtotocytotoxic compound is a compound selected from the group consisting of a chlorin e6 derivative, a cyanine derivative, a bacteriochlorin derivative, a squaraine derivative, or a phthalocyanine derivative.

22. The method of claim 14, wherein the target cell is at least one of a bacteria, a fungus, a protozoa, an amoeba, a parasitic organism, a cancer cell, fat cell, biofilm, vascular plaque, and a cancer cell.

23. The method of claim 14, wherein the photocytotoxic compound possesses at least one of limited photostability and photo-oxidation stability.

24. The method of claim 14, wherein the selective photoluminescent compound possesses at least one of photostability and photo-oxidation stability.

25. A light source comprising:
a light pathway configured to transmit a light of a first wavelength; and
a tip section having a photoluminescent material located along the light pathway, the light of the first wavelength configured to be received by the photoluminescent material of the tip section and emitted from the light source as an emitted light having a second wavelength.

26. The light source of claim 25, further comprising a reflective coating configured to direct the light of the first wavelength to the photoluminescent material.

27. The light source of claim 25, further comprising an end cap at an end of the tip section, the end cap configured to redirect the light back to the tip section.

28. The light source of claim 25, further comprising a photonic lattice array imprinted onto the tip section and configured to narrow the emission spectrum of the emitted light.

29. The light source of claim 25, further comprising a medical compatible coating encapsulating the tip section.
